(19) United States
(12) Patent Application Publication
(10) Pub. No.: US 2019/0086402 A1
(43) Pub. Date: Mar. 21, 2019

(54) BIOSENSOR AND METHOD FOR ANALYZING SPECIMEN BY USING SAME

(71) Applicant: PLEXENSE, INC., Davis, CA (US)

(72) Inventors: Gibum Kim, Daejeon (KR); Taeyoung Kim, Seongnam-si (KR); Chealkon Kim, Busan (KR); Hyejin Hwang, Yongin-si (KR); Jiyoung Lee, Gyeryong-si (KR)

(21) Appl. No.: 16/195,666

(22) Filed: Nov. 19, 2018

Related U.S. Application Data
(63) Continuation-in-part of application No. PCT/KR2017/004546, filed on Apr. 28, 2017.

(30) Foreign Application Priority Data
May 17, 2016 (KR) 10-2016-0060161

(51) Int. Cl. G01N 33/543; G01N 21/552; B01L 3/00

(52) U.S. Cl. CPC G01N 33/54373; G01N 21/554; B01L 3/508; B01L 2200/026; B01L 2300/123; B01L 2300/0636; B01L 2400/0406; B01L 2300/042; B01L 2300/0609

(57) ABSTRACT

The disclosed technology relates to a biosensor and an analysis method using the same. The biosensor includes a sensing unit and a gripping unit. The sensing unit includes a substrate having a thin film layer formed on one or more surfaces thereof, wherein the thin film layer comprises conductive nanoparticles or nanostructures that are configured such that localized surface plasmon resonance (LSPR) is induced in response to light incident thereon. When immersed in a target sample containing a target analyte, the thin film layer is configured to bind thereon the target analyte. The gripping unit connected to one end of the substrate and configured to be gripped by a user.

### CROSS-REFERENCE TO RELATED APPLICATIONS

This application is a continuation in part of PCT Application No. PCT/KR2017/004546, filed on Apr. 28, 2017, which claims priority to Korean Patent Application No. KR 10-2016-0060161, filed on May 17, 2016. Each of the above applications is incorporated herein by reference in its entirety.

### BACKGROUND

### Field

The disclosed technology relates to a biosensor and a sample analysis method using the biosensor.

### Description of the Related Technology

Common methods for analyzing biological samples such as nucleic acids and proteins can be divided into two major areas as follows. The first area includes methods of analyzing the concentration of a sample by measuring optical absorbance using an ultraviolet-visible spectroscopic method. In the method, an absorbance is measured by passing light of a certain intensity through a sample and then comparing intensity of light before and after the passage. Such an optical absorbance measurement method measures the concentration of a specific functional group contained in the sample. Therefore, there exists inconvenience of applying one or more additional analytical methods in order to quantitatively analyze the reactivity and activity of a specific binding substance in a biological reaction. Furthermore, the method offers a low analytical sensitivity of 10⁻⁶M and thus it is not suitable for analyzing a biological sample that typically requires an analytical sensitivity as high as or higher than 10⁻¹²M.

The second area includes methods that utilize enzyme immunoassay. Enzyme immunoassay is a method commonly used for quantitatively analyzing the reactivity and activity of a specific sample at an analytical sensitivity as high as or higher than 10⁻¹²M. The enzyme immunoassay uses a quantitative analysis method in which a sample is analyzed using an enzyme-labeled antibody formed by chemical binding of an enzyme such as peroxidase or galactosidase with an antibody in a target-specific antigen-antibody reaction. Alternatively, fluorescence immunoassay can be used in which a sample is analyzed using an antigen or antibody labeled with a fluorescent dye such as fluorescein and rhodamine and a fluorescence analyzer.

These analytical methods are widely used because they permit to analyze, with an excellent detection sensitivity, the reactivity and activity of the reaction between a reactant and a target analyte in a sample. However, they still have problems of a long assay time and high assay cost because of complicated sample processing, labeling of a sample or target analyte with a fluorescent dye, and/or use of an expensive analyzer. In particular, enzyme immunoassay or fluorescence immunoassay has difficulties in rapid screening of a large number of libraries during drug development or biomarker development due to a long assay time and necessity of using a separate target-specific antibody depending on the target analyte.

Therefore, there is a desperate need for a solution to the problem of the conventional sample analysis method.

### SUMMARY

The disclosed technology aims to solve the aforementioned problems as well as other problems of the conventional arts. One aspect of the disclosed technology is to form a thin film layer in which conductive nanoparticles or nanostructures are dispersedly disposed on one or more surfaces of the substrate and to provide a biosensor that detects a sample by inducing an LSPR phenomenon.

A different aspect of the disclosed technology is to provide a biosensor that has a narrow portion having a relatively narrow width at a predetermined position of a substrate so as to prevent a sample from rising along a gap between two immediate substrates or between a substrate and an inner wall of a cuvette.

Another aspect of the disclosed technology is to provide a relatively simple and inexpensive sample analysis method using a biosensor utilizing an LSPR phenomenon without a separate sample pre-treatment process.

A biosensor according to an embodiment of the disclosed technology comprises a substrate having a predetermined length and a thin film layer, formed by dispersing and arranging conductive nanoparticles or nanostructures on at least one of the both sides of the substrate to cause localized surface plasmon resonance (LSPR) phenomenon, which is immersed in a target sample to bind a target analyte in the target sample; and a gripping unit connected to one end of the substrate and gripped by a user.

In addition, a biosensor according to an embodiment of the disclosed technology further comprises a cap connected to the substrate and the gripping unit and releasably inserted into a cuvette accommodating the target sample.

In addition, a biosensor according to an embodiment of the disclosed technology further comprises a fixing unit which is arranged on an outer surface of the cap and has deformable resilience capable of being close contact with an inner circumferential surface of the cuvette when the cap is inserted into the cuvette.

In addition, in a biosensor according to an embodiment of the disclosed technology, the fixing unit is formed to be extended and bent from an outer surface of the cap.

In addition, in a biosensor according to an embodiment of the disclosed technology, an outer surface portion of the cap facing the fixing unit is recessed.

In addition, in a biosensor according to an embodiment of the disclosed technology the substrate comprises a narrow portion having a relatively narrow width at a predetermined height with respect to the other end of the substrate.

In addition, in a biosensor according to an embodiment of the disclosed technology, the narrow portion is formed as an ascend prevention groove dented concavely from the side of the substrate.

In addition, in a biosensor according to an embodiment of the disclosed technology, the ascend prevention grooves are formed on both sides of the substrate.

In addition, in a biosensor according to an embodiment of the disclosed technology, a plurality of the ascend prevention grooves are formed along the sides and spaced apart in the lengthwise direction of the substrate.

In addition, in a biosensor according to an embodiment of the disclosed technology, a plurality of the sensing units are spaced apart from one another and placed side by side.

In addition, a biosensor according to an embodiment of the disclosed technology further comprises a pair of guards, disposed opposite to each other with the sensing unit therebetween, to protect the sensing part.

In addition, a biosensor according to an embodiment of the disclosed technology comprises an adaptor, disposed under bottom surface of a cuvette accommodating the target sample thereinto, to adjust a height of the cuvette.

In addition, in a biosensor according to an embodiment of the disclosed technology, the adaptor is formed in a block shape, and comprises an engaging groove on the outer surface of the adaptor for binding to a bottom surface of the cuvette.

In addition, in a biosensor according to an embodiment of the disclosed technology, the sensing unit is immersed in the target sample, and then the target sample is analyzed by irradiating the cuvette with light.

In addition, in a biosensor according to an embodiment of the disclosed technology, the analysis of the target sample is protein assay, immunoassay, kinetic analysis, or small molecule detection.

In another aspect, a sample analysis method using a biosensor according to an embodiment of the disclosed technology comprises (a) preparing the biosensor according to any one of the claims 1 to 15; (b) immersing a sensing unit of the biosensor in a detection sample containing a detection substance and immobilizing the detection substance; and (c) immersing the sensing unit in a target analyte by inserting the sensing unit on which the detection substance is immobilized in a cuvette accommodating the target analyte which specifically binds to the detection substance.

In addition, a sample analysis method using a biosensor according to an embodiment of the disclosed technology may further comprise a step of immersing the sensing unit of the biosensor in a rinsing solution between the steps (b) and (c).

In addition, a sample analysis method using a biosensor according to an embodiment of the disclosed technology may further comprise a step of measuring absorbance, after the (c), by arranging the biosensor of which the sensing unit is inserted in the cuvette in a spectroscopic analyzer.

In addition, in a sample analysis method using a biosensor according to an embodiment of the disclosed technology the sensing unit is inserted and immersed in a cuvette containing the detection substance in the (c), and may further comprise a step of measuring absorbance by placing the biosensor of which the sensing unit is immersed in the cuvette containing the detection substance into a spectroscopic analyzer between the steps (b) and (c).

In addition, in a sample analysis method using a biosensor according to an embodiment of the disclosed technology may further comprise a step of measuring absorbance by placing the biosensor of which the sensing unit is inserted and immersed in a cuvette containing a rinsing solution into a spectroscopic analyzer.

### BRIEF DESCRIPTION OF THE DRAWINGS

FIG. 1 is a perspective view of a biosensor according to an embodiment of the disclosed technology.

FIG. 2A is a cross-sectional view taken along the line A-A′ of the biosensor illustrated in FIG. 1 where a thin film layer is formed on one but not the other of opposing major surfaces of the substrate.

FIG. 2B is a cross-sectional view taken along the line A-A′ of the biosensor illustrated in FIG. 1 where a thin film layer is formed on both of opposing major surfaces of the substrate.

FIG. 3 is a side view of a biosensor which is inserted into a cuvette, according to an embodiment of the disclosed technology.

FIG. 4 is a front view of a biosensor which is inserted in a cuvette, according to an embodiment of the disclosed technology.

FIG. 5 is a perspective view of a biosensor according to another embodiment of the disclosed technology.

FIG. 6 is a perspective view of an adaptor according to an embodiment of the disclosed technology.

FIG. 7 is a graph showing a signal of a biosensor according to an embodiment of the disclosed technology.

FIG. 8 is a flowchart illustrating a method of analyzing a sample using a biosensor according to an embodiment of the disclosed technology.

FIG. 9 is a graph of absorbance change analyzed by a sample analysis method using a biosensor according to an embodiment of the disclosed technology.

### DETAILED DESCRIPTION

To address various needs of analyzing biological samples, a biosensor and an assay method based on localized surface plasmon resonance (LSPR) induced in the biosensor is disclosed. In the disclosed LSPR-based method, a sample concentration-dependent refractive index change is analyzed by measuring a change in the intensity or wavelength of light reflected from or transmitted through a metal-containing thin film layer, e.g., a thin film layer formed by coating metal nanoparticles on a substrate surface, e.g., which may be transparent, while irradiating the thin film layer with light. Analytical methods utilizing such an LSPR phenomenon for assaying biological or non-biological samples have been investigated to overcome disadvantages associated with existing fluorescence-based analysis methods, such as complex sample processing and long analysis time.

The features and advantages of the disclosed technology will become more apparent from the following description with reference to the appended drawings.

It should be understood that some terms and words used in the present specification and the claims are not to be construed as having common and dictionary meanings but are to be construed as having meanings and concepts corresponding to the technical spirit of the present disclosure in view of the principle that the inventor can define properly the concept of the terms and words in order to describe his/her invention with the best method.

A biosensor of the disclosed technology quantitatively detects a sample by inducing an LSPR phenomenon on the thin film layer of metal nanoparticles or nanostructures dispersedly disposed on one or more surfaces of the substrate. The biosensor is configured to facilitate the reaction between biological samples or between biological and non-biological samples, without a separate sample pre-treatment process.

In addition, in a biosensor of the disclosed technology, the sensing unit is configured to be immersed in a cuvette containing a sample, for analyzing the sample and has a narrow portion having a relatively narrow width formed at a predetermined position of the substrate constituting the sensing unit. The narrow portion offers an advantage of preventing the sample from rising by a capillary force along a gap between two immediately adjacent substrates in parallel or between the substrate and the inner surface of the cuvette.

In addition, a sample analysis method using a biosensor of the disclosed technology is based on a LSPR phenomenon and thus may not include chromophore labelling, unlike enzyme immunoassay that include a relatively complicated step of labelling a sample molecule with a fluorescent dye. Therefore, the biosensor permits to quantitatively analyze a sample through a simple detection process only with a visible light spectroscopic analyzer.

The objectives, specific advantages, and novel features of the disclosed technology will become more apparent from the following detailed description and preferred embodiments with reference to the appended drawings. It should be noted that the same reference numerals are denoted to the elements of the drawings in the present specification with the same numerals as possible, even if they are displayed in other drawings. Also, the terms “the first”, “the second” and the like are used to distinguish one element from another and thus the element is not limited thereto. Hereinafter, in the description of the disclosed technology, a detailed explanation of related known arts which may unnecessarily obscure the gist of the disclosed technology will be omitted.

Hereinafter, preferred embodiments of the disclosed technology will be described in detail with reference to the appended drawings.

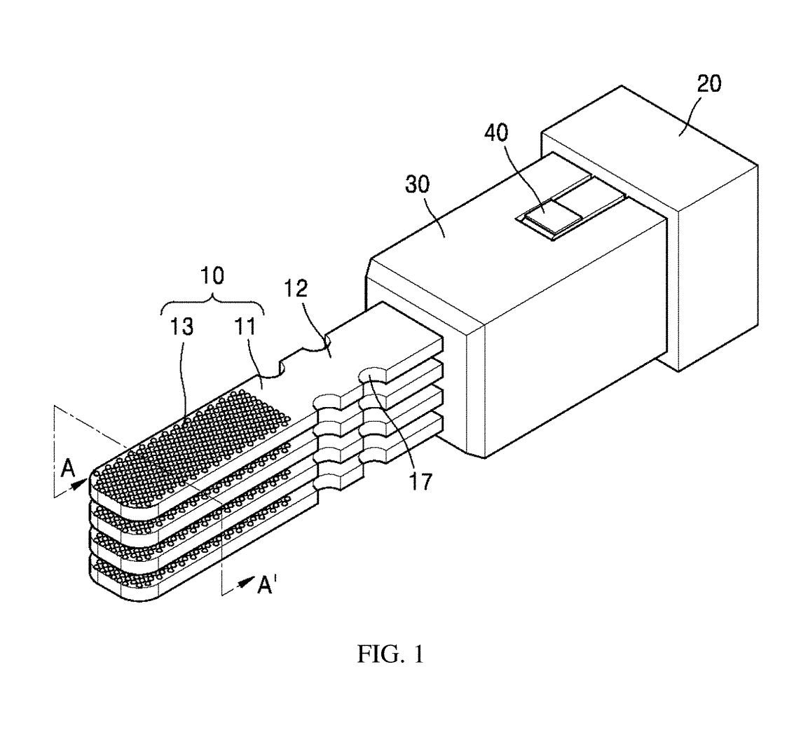

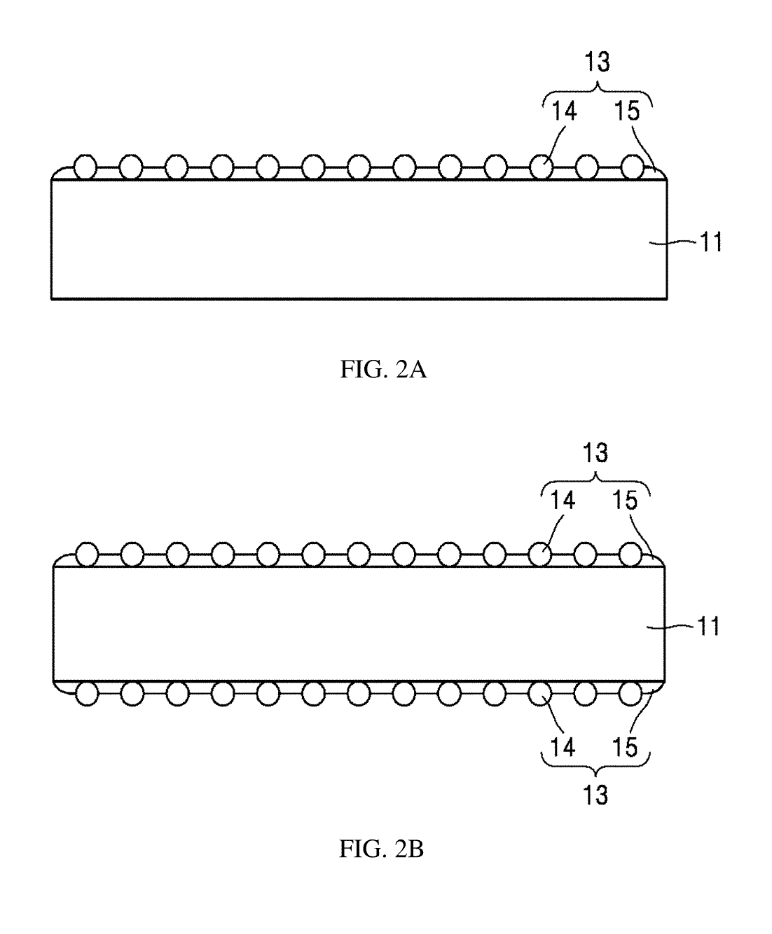

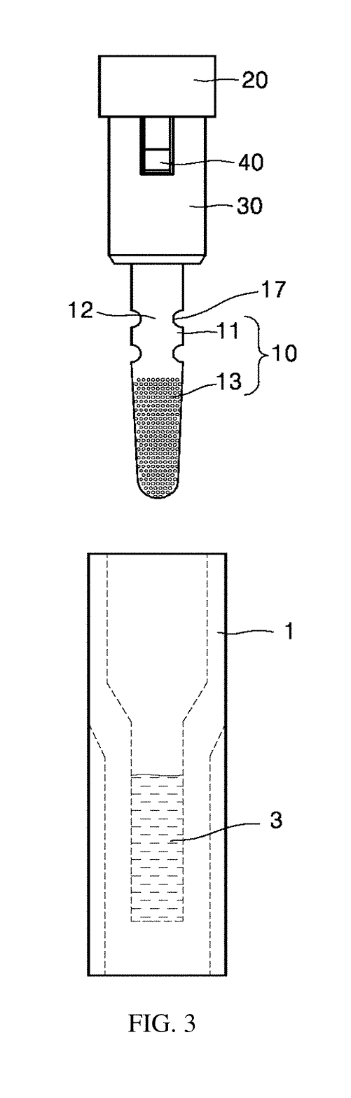

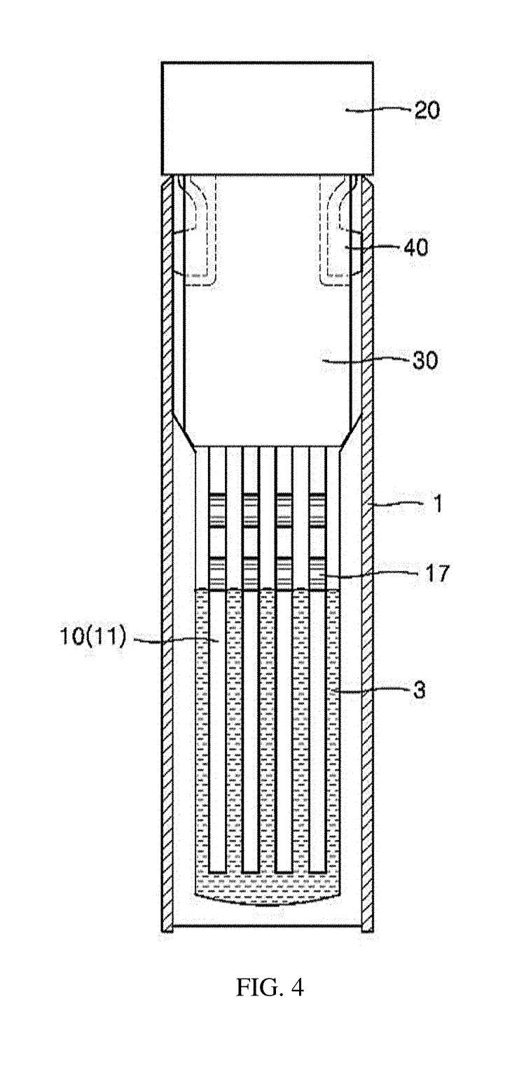

FIG. 1 is a perspective view of a biosensor according to an embodiment of the disclosed technology; FIG. 2A is cross-sectional view taken along the line A-A′ of FIG. 1 where a thin film layer is formed on one but not the other of opposing major surfaces of the substrate; FIG. 2B is a cross-sectional view taken along the line A-A′ of the biosensor in FIG. 1 where a thin film layer is formed on both of opposing major surfaces of the substrate; FIG. 3 is a side view of a biosensor inserted into a cuvette according to an embodiment of the disclosed technology; FIG. 4 is a front view of a biosensor inserted into a cuvette according to an embodiment of the disclosed technology.

As illustrated in FIGS. 1 to 4, a biosensor according to an embodiment of the disclosed technology comprises a substrate 11 having a predetermined length; a sensing unit 10 having a thin film layer 13 formed by dispersedly disposing conductive nanoparticles or nanostructures 14 on at least one of the both surfaces of the substrate 11 to induce an LSPR phenomenon and being immersed in a target sample 3 to induce a reaction between a target analyte in the target sample 3 and a surface of the thin film layer 13; and a gripping unit 20 connected to one end of the substrate 11 and gripped by a user.

A biosensor according to an embodiment of the disclosed technology relates to a sensor for detecting a sample utilizing an LSPR phenomenon, and comprises a sensing unit 10 and a gripping unit 20.

Surface plasmon resonance (SPR) refers to a phenomenon of the propagation of surface plasmon polaritons which are generated on or near the surface of conductive materials by coupling of electrons and photons having a specific wavelength. Without being bound to any theory, SPR refers to a phenomenon of the collective oscillation of conduction band electrons propagating along the interface between a metal with a negative dielectric constant and a medium with a positive dielectric constant. SPR results in a reflected or transmitted electromagnetic wave having an enhanced intensity in comparison with an incident electromagnetic wave and shows characteristics of an evanescent wave which exponentially decays with increasing distance away from the interface in a direction perpendicular to the interface.

SPR can be classified as a propagating surface plasmon resonance (PSPR) observed at the interface between a dielectric material and a 10-200 nm-thick flat metal surface; and localized surface plasmon resonance (LSPR) observed from nanoparticles or nanostructures. A biosensor based on LSPR detects a change in the LSPR wavelength showing a maximum absorption or scattering which depends on a change of the chemical and physical environment on the surface (for example, a change in refractive index of a medium near the surface) of the nanoparticles or nanostructure. The detection of the LSPR wavelength change permits to distinguish specific molecules or to analyze concentration of specific molecules in a medium. LSPR is highly sensitive to the change of refractive index and that allows label-free detection. A biosensor according to the disclosed technology is fabricated such that the LSPR phenomenon can be applied.

The biosensors according to various embodiments are configured such that LSPR occurs in the sensing part 10, which comprises the substrate 11 and the thin film layer 13.

Here, the substrate 11 includes a plate-shaped part having a predetermined length. The substrate 11 is not necessarily limited to a flat plate, but may be formed in various shapes such as a curved shape or a convexo-concave ( [symbol] ) shape. However, hereinafter, the description assumes a flat plate.

In some embodiments, the substrate 11 may be an optically transparent or opaque substrate 11. When the biosensor is configured such that light transmitted through the substrate is detected, an optically transparent substrate 11 is preferable. However, when the biosensor is configured such that light reflected from the substrate is detected, the substrate 11 may be optically transparent or opaque. It will be appreciated that, even when light transmitted through the substrate is detected, it is not necessary that the entire the substrate 11 be optically transparent. Instead, the substrate 11 may include optically transparent portions that are configured to transmit light, and include other portions that may be opaque. The optically transparent substrate 11 may include, for example, glass or a polymer material having a certain degree of optical transparency. The polymer material may comprise polycarbonate (PC), polyethylene terephthalate (PET), polymethyl methacrylate (PMMA), triacetyl cellulose (TAC), cycloolefin, polyarylate, polyacrylate, polyethylene naphthalate, polybutylene terephthalate or polyamide. However, the polymer material is not necessarily limited thereto. A transparent substrate 11 may comprise sapphire. An opaque substrate 11 may comprise silicon, e.g., a single crystal. However, the material of the substrate 11 is not limited to the aforementioned materials and various other materials can be utilized in consideration of the conditions of the target analyte, the fabrication process, and the like.

Referring to FIGS. 2A and 2B, the thin film layer 13 is a layer formed on one or more surfaces of the substrate 11, e.g., one or both of opposing major surfaces of the substrate 11, and is formed by dispersedly disposing conductive nanoparticles or nanostructures 14 that cause LSPR. The thin film layer 13 may be formed on only one of opposing major surfaces (see FIG. 2A) of the substrate 11 or on both of opposing major surfaces (see FIG. 2B) of the substrate 11. Embodiments are not so limited. For example, while not illustrated, the thin film layer 13 may be formed on one or both of side surfaces in some implementations. In addition, while the illustrated substrate 11 is a planar substrate having opposing major surfaces, embodiments are not so limited and can have any suitable shape, such as any polygonal or nonpolygonal shapes.

The conductive nanoparticles or nanostructures 14 may have any shape selected from a nanosphere, a nanotube, a nanocolumn, a nanorod, a nanopore, a nanowire, or combinations thereof. The nanoparticles or nanostructures may be completely filled, porous or hollowed depending on the shape. The conductive nanoparticles or nanostructures 14 may be conductive particles of carbon, graphite, metalloid, metal, metalloid alloy, metal alloy, conductive metal oxide, conductive metal nitride; or core-shell structure particles in which a conductive layer such as a metal thin film is coated on an insulating core. However, the conductive nanoparticles or nanostructures 14 are not necessarily limited to the aforementioned shapes and materials.

In some embodiments, the nanoparticles or nanostructures 14 may be dispersed randomly and may have a relatively wide distribution of inter-particle distances. In some other embodiments, the nanoparticles or nanostructures 14 may be patterned structures having a relatively narrow distribution of inter-particle distances. However, regardless of whether or not the nanoparticles or nanostructures 14 are patterned, the nanoparticles or nanostructures 14 have sizes, shapes and inter-particle spacings that are adapted for LSPR-based detection described herein.

The conductive nanoparticles or nanostructures 14 may be immobilized (see FIGS. 2A and 2B) on the substrate 11 by a binder 15 wherein the binder 15 may be an ionic polymer such as poly diallydimethylammonium chloride, poly allylamine hydrochloride, poly 4-vinylbenzyltrimethyl ammonium chloride, polyethyleneimine, poly acrylic acid, poly sodium 4-styrene sulfonate, poly vinylsulfonic acid, poly sodium salt, poly amino acids or a mixture thereof. However, the binder 15 is not limited to the aforementioned polymers, as long as it is a material capable of immobilizing nanoparticles or nanostructures 14 on the substrate 11.

Referring to FIG. 3, in operation, the sensing unit 10 formed by disposing the thin film layer 13 on the substrate 11 is immersed in the target sample 3. At this time, the thin film layer 13 is also immersed in the target sample 3 as the substrate 11 is immersed from its free end in the target sample 3. Here, the free end of the substrate 11 refers to the opposite end of one end of the substrate 11 connected to the gripping unit 20 to be described later. When the thin film layer 13 is immersed in the target sample 3, a target analyte in the target sample 3 binds to the thin film layer 13.

At this time, a detection substance that specifically binds with the target analyte may be immobilized on the thin film layer 13 in order for the thin film layer 13 to bind with the target analyte. The detection substance may be, for example, a low molecular weight compound, an antigen, an antibody, a protein, a peptide, a DNA, an RNA, a PNA, an enzyme, an enzyme substrate, a hormone receptor, and a synthetic reagent having a functional group. However, the aforementioned detection substances are just exemplary ones and the detection substance is not necessarily limited thereto. The detection substance may comprise any suitable substances, including combinations of such substances, that can combine with the target analyte. The detection substance is immobilized on the thin film layer 13, e.g., on the conductive nanoparticles or nanostructures 14, or on the binder 15. The detection substance can specifically bind to the target analyte, thereby binding the target analyte to the thin film layer 13. However, the detection substance is not necessarily immobilized on the thin layer 13.

In some embodiments, the biosensor may comprise one sensing unit 10. In some other embodiments, e.g., the illustrated embodiment with respect to FIG. 1, the biosensor comprises more than one sensing units 10. When a plurality of sensing units 10 are present, adjacent ones of the sensing units 10 are spaced apart from each other by a predetermined distance and arranged side by side. In this case, the sensing units 10 are arranged such that one surface of the substrate 11 faces one surface of another substrate 11. One or more of the sensing units 10 are connected to and fixed to the gripping unit 20.

Various aspects of the biosensor, including the configurations and arrangements of the sensing unit 10, the thin film layer 13, the binder 15 and the conductive nanoparticles or nanostructures 14, are described in U.S. application Ser. No. 14/863,238, filed Sep. 23, 2015, which content is incorporated by reference in its entirety.

The gripping unit 20 is a unit held by a user and is directly or indirectly connected to one end of the substrate 11. Thus, the user can hold the gripping unit 20 and immerse the sensing unit 10 into the cuvette 1 in which the target sample 3 is accommodated. The cuvette 1 is a container configured to accommodate the target sample 3 for the spectroscopic analysis of a target sample 3. However, the target sample 3 is not necessarily accommodated in the cuvette 1 when the sensing unit 10 is immersed. Nevertheless, in the general sample analysis process, the target sample 3 is prepared in the cuvette 1 and the spectroscopic analysis is performed in a state in which the sensing unit 10 is immersed in the cuvette 1. Therefore, hereinafter, the operation of the biosensor may be described based on a configuration in which the target sample 3 is accommodated in the cuvette 1.

In the illustrated embodiment, the gripping unit 20 is a separate unit that is substantially thicker and/or wider than the sensing unit 10 to facilitate gripping by a user. The gripping unit 20 may be formed of a different material than the sensing unit 10. In the illustrated embodiment, the gripping unit 20 is formed separately and is attached to the sensing unit 10, and therefore may not form an integral unit with the sensing unit 10. However, embodiments are not so limited, and in other implementations, the gripping unit 20 may form an extension of the sensing unit 10 and is formed an integral unit with the sensing unit 10, In yet other implementations, the gripping unit 20 is formed as an integral unit with one or more of a cap 30 and a fixing unit 40 described below.

According to the aforementioned descriptions, the biosensor according to the present embodiment has a structure in which the thin film layer 13 having the conductive nanoparticles or nanostructures 14 dispersedly disposed on one surface of the substrate 11 is formed. The thin film layer 13 causes LSPR which can be utilized for identifying the specific target analyte and determining the concentration of the target analyte in a medium. The biosensor allows a label-free detection of the target analyte.

In some embodiments, a biosensor according to the present embodiment may further comprise a cap 30. The cap 30 is configured to be removably inserted into the cuvette 1 and to close or seal the open inlet of the cuvette 1. The cap 30 is disposed under the gripping unit 20 and connects the gripping unit 20 and the substrate 11. The cap 30 is held in contact with the inner surface of the cuvette 1 and fix the substrate 11 so that the sensing unit 10 does not move in the cuvette 1.

The sizes of different cuvettes 1 may not be uniform. Therefore, depending on the size of the cuvette 1, even when the cap 30 is inserted into the cuvette 1, the cuvette 1 may not form a snug fit with the cuvette 1. Instead, a gap may be created between the outer surface of the cap 30 and the inner surface of the cuvette 1 when the cap 30 is inserted. Due to the gap, the cap 30 remains unfixed to the cuvette 1, and the sensing unit 10 may not be securely fixed in position during the analysis, making it difficult to accurately analyze the target sample 3. To avoid this problem, the biosensor may further comprise a fixing unit 40 to fix or secure the sensing unit 10 regardless of the size of the cuvette 1.

The fixing unit 40 is arranged on the outer surface of the cap 30. With this arrangement, the original position or shape of the fixing unit 40 is changed, e.g., deformed, to create resilience or impart an elastic force on an inner surface of a cuvette 1 when the cap 30 is inserted into the cuvette 1. The fixing unit 40 is brought into close contact with the inner circumferential surface of the cuvette 1 by the resilience. The sensing unit 10 connected to the cap 30 is fixed or secured in the cuvette 1 when the fixing unit 40 disposed on the cap 30 is in close contact with the cuvette 1. Thus, the elastic force imparted on the inner surface of the cuvette 1 by the fixing unit 40 may suppress movement of the cap 30 and the sensing unit 10 when inserted into cuvette 1.

Specifically, the fixing unit 40 may be an elastic unit that is deformed while being pressed by the inner surface of the cuvette 1 and comes into close contact with the inner surface of the cuvette 1 by elasticity when the cap 30 is inserted into the cuvette 1. The fixing unit 40 may use the inherent elasticity of the elastic material such as rubber or the like, or may use the properties of a unit such as a spring. However, the fixing unit 40 does not necessarily have to use the elasticity of the elastic material or the unit, but can be implemented through a predetermined structure, which will be described in detail below (see FIG. 4).

The fixing unit 40 may extend from the outer surface of the cap 30 and may be bent in a predetermined direction. For example, the fixing unit 40 may extend outward from the outer surface of the cap 30 and may be bent in parallel to the outer surface of the cap 30 to form an inverted L shape. The outwardly protruding protrusion formed at one end of the fixing unit 40 is pressurized against the inner surface of the cuvette 1, and as a result, the fixing unit 40 can be brought into close contact with the inner surface of the cuvette 1 by tension. At this time, since the fixing unit 40 is moved toward the cap 30 when pressured, a portion of the outer surface of the cap 30 opposite to the fixing unit 40 may be recessed.

Alternatively, the fixing unit 40 may extend from the inner surface of the recessed portion of the cap 30 and the protrusion may be formed outward from the outer surface of the cap 30 to have an “L” shape.

Consequently, the fixing unit 40 may extend from the outer surface of the cap 30 and be freely modified into various structures so long as it can be brought into close contact with the inner surface of the cuvette 1 by tension when the cap 30 is inserted into the cuvette 1.

In some embodiments of a biosensor, the substrate 11 may have a narrow portion 12. Here, the narrow portion 12 is a portion where the width of the substrate 11 is relatively narrowed at a predetermined height with respect to the other end (free end) of the substrate 11, as compared with other portions.

In order to analyze the target sample 3, the cuvette 1 may be irradiated with light from the outside of the cuvette 1 in a state in which the sensing unit 10 is inserted. When the sensing unit 10 is inserted into the cuvette 1 containing a liquid such as the target sample 3, the liquid may flow by capillary action in the gap between the sensing unit 10 and the inner surface of the cuvette 1 or in the gap between the immediately arranged sensing units 10 that are arranged in parallel. The capillary action may cause the target sample 3 in the gap to rise towards the cap 30 (see FIG. 4). Due to the rise of the target sample 3, the target sample 3 moves to a position beyond the region of the substrate 11 on which the thin film layer 13 disposed. This may increase the amount of the target sample 3 used in the analysis and the reliability of the analysis may degrade. The narrow portion 12 may be configured to solve this problem. The target sample 3 rises along the substrate 11 by the attractive force between the target sample 3 and the substrate 11. Thus, when the width of the substrate 11 becomes narrowed in the narrow portion 12, the area of contact between the substrate 11 and the target sample 3 is reduced, so that the target sample 3 is suppressed from rising.

In some implementations, the narrow portion 12 may be formed through the formation of a groove 17, which may be referred to herein as the ascend prevention groove, configured to suppress the target sample 3 from rising due to capillary action. In the illustrated embodiment, the ascend prevention groove 17 comprises a concave recess formed on one or both side surfaces of the substrate 11. Since the ascend prevention groove 17 is recessed by a predetermined depth from one side surface to the other side surface of the substrate 11, the width of the substrate 11, i.e. the distance between the both side surfaces of the substrate 11, at the position where the ascend prevention groove 17 is formed is reduced. The ascend prevention groove 17 may be formed only on one side surface of the substrate 11, or may be formed on both side surfaces of the substrate 11. In the case where the ascend prevention grooves 17 are formed on the both side surfaces of the substrate 11, they may be formed at the same vertical position so as to face each other. However, embodiments are not necessarily limited thereto. For example, the ascend prevention grooves 17 may be staggered in a zigzag pattern. The ascend prevention grooves 17 may be formed in plurality along the side surfaces of the substrate 11 at a predetermined distance in the lengthwise direction.

The ascend prevention groove 17 may be formed to be rounded in shape but it is not necessarily formed in such a shape. The ascend prevention groove 17 may be recessed in any shape so long as the width of the substrate 11 is narrowed. Since there is no particular limitation on the width of the ascend prevention groove 17, e.g., the distance perpendicular to the depth of the ascend prevention groove 17, the width of the ascend prevention groove 17 may range from a predetermined height of the substrate 11 to one end thereof.

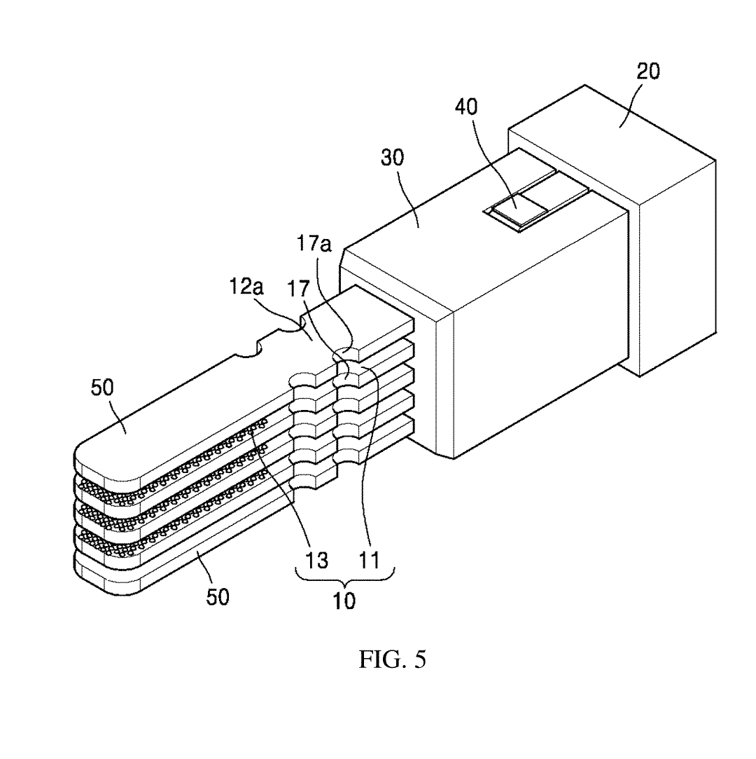

FIG. 5 is a perspective view of a biosensor according to another embodiment of the disclosed technology. As illustrated in FIG. 5, the biosensor according to another embodiment of the disclosed technology may further comprise a pair of guards 50 to protect sensing unit 10. Here, the pair of guards 50 are units that are disposed opposite to each other with the sensing unit 10 therebetween, spaced apart from the sensing unit 10. A pair of the guards 50 is arranged so that one or more sensing units 10 are interposed between the pair of guards 50. Since the guards 50 are disposed on each of the outermost sides of the sensing unit 10, the sensing units 10 are prevented from touching the inner surface of the cuvette 1 and are protected from external impact and the like. The guard 50 may be formed in the same shape as that of the substrate 11, but the shape of the guard 50 is not necessarily limited thereto. However, unlike the sensing unit 10, the thin film layer 13 may be omitted. When the guard 50 is formed in a plate shape, the guard 50 may also be formed with a narrow portion 12a whose width is relatively narrow at a predetermined height, because the sample 3 may rise in the gap between the guard 50 and an adjacent sensing unit 10, which may be arranged in parallel, or between the guard 50 and an adjacent inner surface of the cuvette 1. At this time, the narrow portion 12a may be formed by laterally recessing the side surface of the guard 50 to form the ascend prevention groove 17a. However, embodiments are not so limited and it is not necessarily required to form the narrow portion 12a on the guard 50 or to form the narrow portion 12a by the ascend prevention groove 17a. In addition, when the biosensor is configured to transmit light that is irradiated on the sensing unit 10 during the sample analysis process, the guard 50 is preferably transparent or have transparent portions. However, embodiments are not so limited, and the guard 50 is not necessarily required to be transparent, for example, when the biosensor is configured to reflect light that is irradiated thereon.

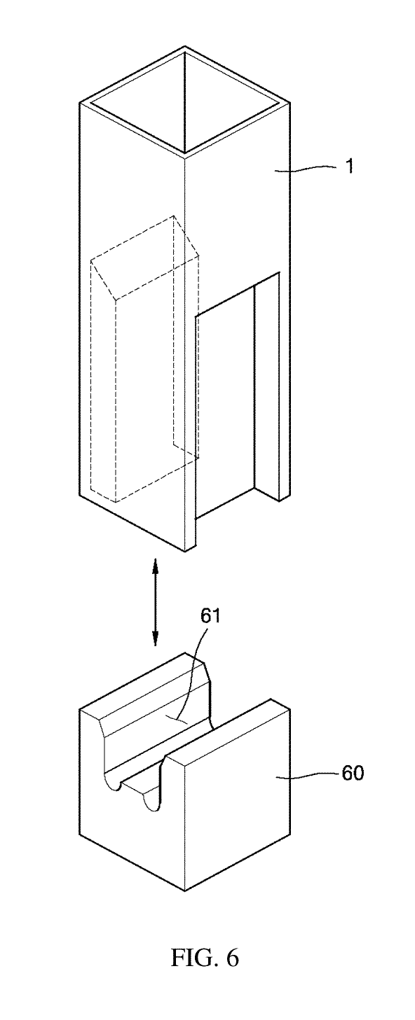

FIG. 6 is a perspective view of an adaptor 60 for a biosensor according to an embodiment of the disclosed technology. As illustrated in FIG. 6, the biosensor according to the present embodiment may further comprise an adaptor 60. Here, the adaptor 60 is a part to adjust the height of the cuvette 1. In order to analyze the target sample 3, the sensing unit 10 is inserted into the cuvette 1 and then light is irradiated from the outside of the cuvette 1 toward the thin film layer 13 of the sensing unit 10 (see FIG. 3). In general, since the height, width, and focus of the light are different depending on the equipment used for light irradiation, the manufacturer of the cuvette 1 designs the height of the cuvette 1 in accordance with the predetermined light irradiation equipment. Therefore, when a cuvette 1 which has a height that is not matched with the path of the light, the cuvette 1 may not be irradiated with light at the proper height for the sample analysis, limiting versatility of the cuvette 1. The height of the cuvette 1 can be adjusted by arranging the adaptor 60 having a suitable shape and dimension under the bottom surface of the cuvette 1 in configurations where the use of the cuvette 1 is problematic because the light irradiation equipment does not match the cuvette 1. Therefore, the position of light irradiation can be conveniently adjusted by controlling the height of the cuvette 1, irrespective of the type of the light irradiation equipment and the cuvette 1.

In the illustrated implementation, the adaptor 60 is formed in a block shape having a predetermined height and has an engaging groove 61 recessed or perforated from the outer surface. The adaptor 60 and the cuvette 1 are combined by inserting the bottom surface of the cuvette 1 into the engaging groove 61. Here, the adaptor 60 can be fabricated to have a different height and combined with the cuvette 1 to adjust the height of the cuvette 1.

The biosensor according to the present embodiment is configured to analyze target sample 3 by irradiating the cuvette 1 with light in a state in which the sensing unit 10 is immersed in the target sample 3, e.g., in a state where the sensing unit 10 is inserted into the cuvette 1. The analysis of the target sample 3 may include one or more of a protein assay, an immunoassay, a kinetic analysis, and small molecule detection. In some conventional techniques, the protein assay, immunoassay, kinetic analysis, and small molecule detection may be performed separately using separate devices. However, the biosensor according to the present embodiment makes it possible to perform one or more of the aforementioned analyses using the same device and/or the same target sample, sequentially or simultaneously.

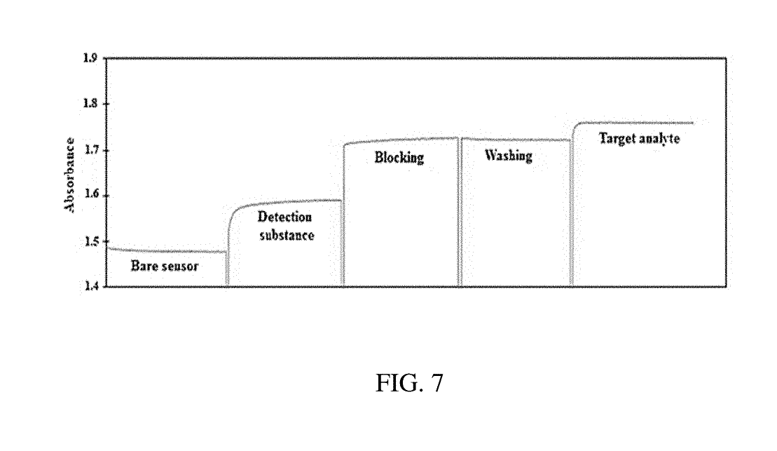

FIG. 7 is a graph showing a signal change measured using a biosensor according to an embodiment of the disclosed technology. FIG. 7 shows a series of signal changes continuously measured under various conditions using the biosensor according to the disclosed technology. In the first step, the background signal of the biosensor according to the disclosed technology was measured in phosphate buffered saline (PBS buffer) solution. In the second step, the biosensor according to the disclosed technology was inserted into the cuvette containing an antibody (detection substance) to measure the signal for antibody immobilization. In the third step, the signal was measured after treating the empty portion (where the detection substance was not immobilized with the antibody) of the metal nanoparticle thin film layer surface with a blocking substance. In the fourth step, extra molecules of the blocking substance not involved in the blocking were removed by washing with a rinsing solution and a signal was measured. In the fifth step, the biosensor according to the disclosed technology was inserted into a cuvette containing an antigen (target analyte) to induce a reaction between the antigen and the immobilized antibody, and the increase of the signal was measured. These observations confirmed a signal change in each step. It was further confirmed that a signal appeared even when the signal was measured after immersing the biosensor according to the disclosed technology into the rinsing solution between the second and third steps (not shown here). From these results, it can be seen that the biosensor according to the disclosed technology operates by showing a specific signal value depending on the substance to be detected.

Hereinafter, a sample analysis method using the biosensor according to the disclosed technology will be described.

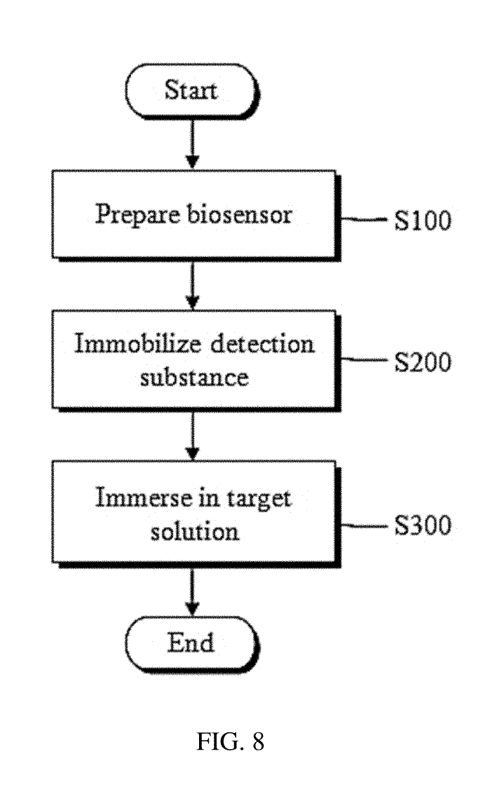

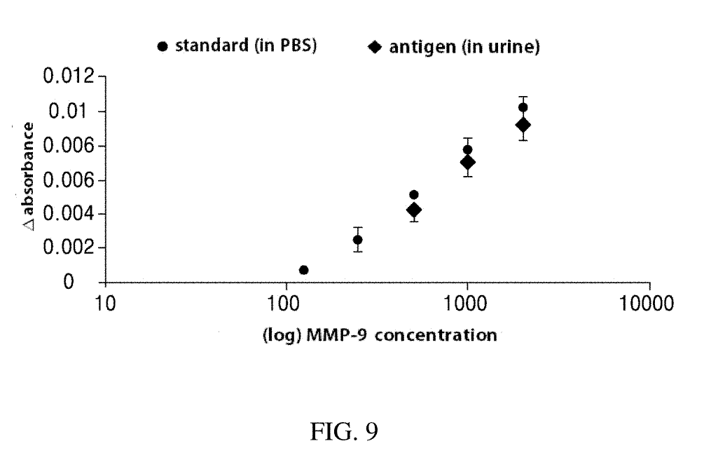

FIG. 8 is a flowchart illustrating a sample analysis method using a biosensor according to an embodiment of the disclosed technology, and FIG. 9 is a graph of absorbance change analyzed by the sample analysis method using a biosensor according to an embodiment of the disclosed technology.

As illustrated in FIG. 8, the sample analysis method using a biosensor according to an embodiment of the disclosed technology comprises: (a) a step of preparing a biosensor according to any one of the above embodiments (S100); (b) a step of immobilizing a detection substance, e.g., on a thin film layer comprising conductive nanoparticles, by immersing the sensing unit of the biosensor in a detection sample containing the detection substance (S200); and (c) a step of immersing the sensing unit, having the detection substance immobilized thereon, of the biosensor in the cuvette containing the target analyte, thereby specifically binding the target analyte to the detection substance (S300).

A sample analysis method using the biosensor according to the present embodiment comprises a step of preparing a biosensor (S100), a step of immobilizing a detection substance (S200), and a step of immersing the sensing unit in a target sample (S300). Here, the sample analysis method using the biosensor according to the present embodiment uses the above-described biosensor according to the disclosed technology, so that redundant contents will be omitted or simply described.

According to the sample analysis method using the biosensor according to the present embodiment, any one of the above-described biosensors is prepared (S100), and the sensing unit of the biosensor is immersed in a detection sample containing a detection substance adapted to specifically bind to the target analyte to immobilize the detection substance on the thin film layer (S200). At this time, the detection substance may be prepared in a cuvette, and then the sensing unit of the biosensor may be immersed in the cuvette. When the biosensor is inserted into the cuvette and the sensing unit is immobilized with the detection substance, the biosensor inserted in the cuvette can be arranged in a spectroscopic analyzer to measure the absorbance. However, the measurement of absorbance here does not necessarily have to be performed.

When the detection substance is immobilized on the sensing unit, the sensing unit is inserted into the cuvette containing the target analyte solution to immerse the sensing unit in the target analyte (S300). At this time, the detection substance of the sensing unit binds to the target analyte. For example, when the detection substance is an antibody and the target analyte is an antigen, an antigen-antibody reaction is induced.

In some implementations, after the detection substance is immobilized on the sensing unit (S200), the sensing unit of the biosensor can be immersed in the rinsing solution before being immersed in the target analyte solution (S300). The detection substance is immobilized to the sensing unit via, for example, a physical or electrostatic reaction. By immersing the sensing unit in the rinsing solution, substances that may be unintended and/or may be attached or bound to the sensing unit in an unintended manner may be removed. At this time, the rinse solution is prepared in a cuvette, and the sensing unit of the biosensor can be inserted into the cuvette to be immersed. When the detection substance is removed, the biosensor inserted in the cuvette can be arranged in a spectroscopic analyzer to measure the absorbance. However, this absorbance measurement is not an essential step to be performed.

After the sensing unit is immersed in the target analyte solution, the target sample can be analyzed by arranging the biosensor in a spectroscopic analyzer while inserting it into the cuvette and then measuring the absorbance. At this time, in some implementations, it may be preferable to pre-heat the spectroscopic analyzer before the biosensor is arranged, and to arrange the biosensor in the spectroscopic analyzer as soon as the sensing unit is immersed in the target analyte solution. However, it is not necessary to pre-heat the spectroscopic analyzer in advance.

The results of analyzing the absorbance difference according to the concentration of the enzyme, MMP-9 (matrix metalloproteinase) in the urine through these steps can be verified in FIG. 9.

Although the disclosed technology has been described herein with reference to the specific embodiments, these embodiments do not serve to limit the invention and are set forth for illustrative purposes. It will be apparent to those skilled in the art that modifications and improvements can be made without departing from the spirit and scope of the invention.

Such simple modifications and improvements of the various embodiments disclosed herein are within the scope of the disclosed technology, and the specific scope of the disclosed technology will be additionally defined by the appended claims.

According to the disclosed technology, it is possible to quantitatively detect a sample by generating an LSPR phenomenon, and to easily induce the reaction between biological samples or between biological and non-biological samples without a separate sample pretreatment process. Therefore, an industrial applicability of the biosensor is recognized.

Although the present invention has been described herein with reference to the specific embodiments, these embodiments do not serve to limit the invention and are set forth for illustrative purposes. It will be apparent to those skilled in the art that modifications and improvements can be made without departing from the spirit and scope of the invention.

Such simple modifications and improvements of the various embodiments disclosed herein are within the scope of the disclosed technology, and the specific scope of the disclosed technology will be additionally defined by the appended Embodiments.

In the foregoing, it will be appreciated that any feature of any one of the embodiments can be combined or substituted with any other feature of any other one of the embodiments.

Unless the context clearly requires otherwise, throughout the description and the claims, the words “comprise,” “comprising,” “include,” “including” and the like are to be construed in an inclusive sense, as opposed to an exclusive or exhaustive sense; that is to say, in the sense of “including, but not limited to.” The word “coupled”, as generally used herein, refers to two or more elements that may be either directly connected, or connected by way of one or more intermediate elements. Likewise, the word “connected”, as generally used herein, refers to two or more elements that may be either directly connected, or connected by way of one or more intermediate elements. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number, respectively. The word “or” in reference to a list of two or more items, that word covers all of the following interpretations of the word: any of the items in the list, all of the items in the list, and any combination of the items in the list.

Moreover, conditional language used herein, such as, among others, “can,” “could,” “might,” “may,” “e.g.,” “for example,” “such as” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or whether these features, elements and/or states are included or are to be performed in any particular embodiment.

While certain embodiments have been described, these embodiments have been presented by way of example only, and are not intended to limit the scope of the disclosure. Indeed, the novel apparatus, methods, and systems described herein may be embodied in a variety of other forms; furthermore, various omissions, substitutions and changes in the form of the methods and systems described herein may be made without departing from the spirit of the disclosure. For example, while features are presented in a given arrangement, alternative embodiments may perform similar functionalities with different components and/or sensor topologies, and some features may be deleted, moved, added, subdivided, combined, and/or modified. Each of these features may be implemented in a variety of different ways. Any suitable combination of the elements and acts of the various embodiments described above can be combined to provide further embodiments. The various features and processes described above may be implemented independently of one another, or may be combined in various ways. All possible combinations and subcombinations of features of this disclosure are intended to fall within the scope of this disclosure.

What is claimed is:

1. A biosensor comprising:
a sensing unit comprising a substrate having a thin film layer formed on one or more surfaces thereof, wherein the thin film layer comprises conductive nanoparticles or nanostructures that are configured such that localized surface plasmon resonance (LSPR) is induced in response to light incident thereon, and wherein when immersed in a target sample containing a target analyte, the thin film layer is configured to bind thereon the target analyte; and
a cuvette configured to hold a target sample and to receive the substrate therein,
wherein when the substrate is inserted into the cuvette holding the target sample, a main surface of the substrate and an adjacent surface form a gap having a width adapted to induce an upper surface of the target sample to rise by capillary force, and
wherein the substrate comprises a narrow portion at a predetermined height with respect to a bottom end of the substrate and configured such that the upper surface of the target sample is suppressed from rising above the narrow portion when the cuvette holds the target sample.

2. The biosensor according to claim 1, wherein the narrow portion is formed by a laterally recessed groove formed at a side surface of the substrate.

3. The biosensor according to claim 2, wherein the narrow portion is formed by a pair of laterally recessed grooves formed on opposing side surfaces of the substrate at the same vertical level.

4. The biosensor according to claim 3, wherein a plurality narrow portions are formed by a plurality of pairs of laterally recessed grooves, wherein the pairs of laterally recessed grooves are spaced apart in a lengthwise direction of the substrate.

5. The biosensor according to claim 1, wherein the gap is formed by the main surface of the substrate and a main surface of the cuvette.

6. The biosensor according to claim 1, wherein the biosensor comprises a plurality of sensing units that are spaced apart from one another and placed side by side, wherein each of the sensing units comprises the substrate having the thin film layer.

7. The biosensor according to claim 6, wherein the gap is formed by the main surface of the substrate and a main surface of an adjacent substrate of an adjacent sensing unit.

8. The biosensor according to claim 1, further comprising a gripping unit connected at a top end of the substrate and configured to be gripped by a user.

9. The biosensor according to claim 8, further comprising a cap connected to the substrate and the gripping unit and is configured to be releasably inserted into a cuvette accommodating the target sample.

10. The biosensor according to claim 9, further comprising an elastically deformable fixing unit formed on an outer surface of the cap and configured to impart an elastic force on an inner circumferential surface of the cuvette to suppress movement of the sensing unit when the cap is inserted into the cuvette.

11. The biosensor according to claim 10, wherein the fixing unit is configured to be extended and bent from an outer surface of the cap.

12. The biosensor according to claim 11, wherein an outer surface portion of the cap facing the fixing unit is recessed.

13. The biosensor according to claim 1, further comprising a pair of guards that are disposed on opposite sides of the sensing unit with the sensing unit disposed therebetween, wherein the pair of guards are configured to protect the sensing unit from contacting surfaces of a cuvette when the sensing unit is inserted into the cuvette.

14. The biosensor according to claim 13, further comprising an adaptor, wherein the adaptor is configured to be coupled to the cuvette at a bottom portion of the cuvette accommodating the target sample, such that a height of the cuvette is upwardly adjusted.

15. The biosensor according to claim 14, wherein the adaptor is formed in a block shape, and comprises an engaging groove on the outer surface of the adaptor for coupling to a bottom surface of the cuvette.

16. The biosensor according to claim 1, wherein the biosensor is configured such that the target sample is analyzed by irradiating light to the cuvette while the sensing unit is immersed in the target sample.

17. The biosensor according to claim 16, wherein the biosensor is configured for analysis of the target sample as part of a protein assay, an immunoassay, a kinetic analysis, or a small molecule detection.

18. A sample analysis method using a biosensor, the method comprising;
(a) preparing the biosensor according to claim 1;
(b) immersing the sensing unit in a detection sample containing a detection substance and immobilizing the detection substance on the sensing unit; and
(c) immersing the sensing unit in the target analyte by inserting the sensing unit on which the detection substance is immobilized in a cuvette accommodating the target analyte, thereby specifically binding the target analyte to the detection substance.

19. The analysis method according to claim 18, further comprising immersing the sensing unit of the biosensor in a rinsing solution between the steps (b) and (c).

20. The analysis method according to claim 18, further comprising measuring absorbance, after the step (c), by placing the biosensor having the sensing unit inserted in the cuvette in a spectroscopic analyzer.

21. The analysis method according to claim 18, wherein the sensing unit is inserted and immersed in the cuvette containing the detection substance in the step (b), wherein the method further comprises measuring absorbance by placing the biosensor having the sensing unit immersed in the cuvette containing the detection substance into a spectroscopic analyzer between the steps (b) and (c).

22. The analysis method according to claim 18, further comprising measuring absorbance by placing the biosensor having the sensing unit inserted and immersed in the cuvette containing a rinsing solution into a spectroscopic analyzer.
